(19) United States
(12) Patent Application Publication
(10) Pub. No.: US 2026/0020821 A1
(43) Pub. Date: Jan. 22, 2026

(54) MAGNETIC ALIGNMENT OF INTRALUMINAL SENSING DEVICE IN CONNECTOR AND ASSOCIATED DEVICES, SYSTEMS, AND METHODS

(71) Applicant: KONINKLIJKE PHILIPS N.V., EINDHOVEN (NL)

(72) Inventor: Miranda TAYLOR, OCEANSIDE, CA (US)

(21) Appl. No.: 19/106,166

(22) PCT Filed: Aug. 16, 2023
(86) PCT No.: PCT/EP2023/072498

§ 371 Date: Feb. 24, 2025

Related U.S. Application Data
(60) Provisional application No. 63/401,741, filed on Aug. 29, 2022.

(51) Int. Cl. A61B 5/00; A61B 5/0215; A61B 5/026; A61M 25/09

(52) U.S. Cl. CPC A61B 5/6851; A61B 5/0215; A61B 5/026; A61B 2560/0443; A61B 2562/227; A61M 25/09

(57) ABSTRACT

A system includes an intravascular guidewire and a connector. The intravascular guidewire includes a flexible elongate member, a sensor, and a guidewire electrical contact. The connector is a connector removably coupled to the intravascular guidewire. The connector includes a slot configured to receive the flexible elongate member and a connector electrical contact. The guidewire and/or the connector comprises a magnet. The magnet facilitates at positioning the proximal portion of the flexible elongate member within the slot and/or aligning the guidewire electrical contact and the connector electrical contact. The connector can include a locking feature. The connector can include a magnet proximate to the locking feature. The guidewire magnet and/or the connector magnet facilitate positioning the flexible elongate member within the slot.

### TECHNICAL FIELD

The subject matter described herein relates to intraluminal physiology sensing devices (e.g., an intravascular pressure sensing and/or flow sensing guidewire). For example, the intraluminal device may include magnetic alignment of a proximal end of the sensing guidewire in a connector.

### BACKGROUND

Intraluminal physiology sensing devices may be introduced into a body lumen of a patient and may, for example, include physiological sensors at a distal end of a catheter or guidewire. Small-diameter medical devices such as intraluminal (e.g., intravascular) catheters and guidewires may incorporate sensors (e.g., pressure, temperature, flow, or imaging sensors) whose power and communications occur through multi-filar (e.g., bifilar, trifilar, etc.) electrical conductor bundle or flat metal ribbons. Electrical wires may be employed to couple such sensors at the distal end of the catheter or guidewire with a connector at a proximal end of the catheter or guidewire. For such catheters and guidewire, segments of electrical contacts are usually arranged at the proximal portion of the guidewire. Proper alignment between electrical contacts of a connector and the electrical connects at the proximal portion of the guidewire are necessary to ensure reliable electrical connection.

The information included in this Background section of the specification, including any references cited herein and any description or discussion thereof, is included for technical reference purposes only and is not to be regarded as subject matter by which the scope of the disclosure is to be bound.

### SUMMARY

Disclosed are intraluminal physiology sensing devices (e.g., an intravascular pressure-sensing and/or flow-sensing guidewire) that includes magnets for aligning a flexible elongate member in a connector. The magnets may be advantageously placed at locations in the connection portion of the flexible elongate member of the sensing guidewire and/or the connector. The location of the magnets pull the flexible elongate member down into the recess of the connector and positions the flexible elongate member such that the locking section is aligned within the slot and the conductive portions are longitudinally aligned with the split, open-comb electrical contacts of the connector. This may provide for correct usage of the locking core feature within the connector, while reducing the risk of misconnection and damage to the proximal end of the flexible elongate member.

In an exemplary aspect, a system is provided. The system includes an intravascular guidewire comprising: a flexible elongate member configured to be positioned within a blood vessel of a patient, wherein the flexible elongate member comprises a proximal portion and a distal portion; a sensor disposed at the distal portion of the flexible elongate member, wherein the sensor is configured to obtain medical data related to the blood vessel while the flexible elongate member is positioned within the blood vessel; a guidewire electrical contact disposed at the proximal portion of the flexible elongate member, wherein the guidewire electrical contact is in electrical communication with the sensor; a connector configured to be removably coupled to the intravascular guidewire, wherein the connector comprises: a slot configured to receive the proximal portion of the flexible elongate member; and a connector electrical contact configured to contact the guidewire electrical contact to establish electrical communication with the sensor when the proximal portion of the flexible elongate member is received within the slot, wherein at least one of the proximal portion of the guidewire or the connector comprises a magnet, wherein the magnet is configured to facilitate at least one of: positioning the proximal portion of the flexible elongate member within the slot; or aligning the guidewire electrical contact and the connector electrical contact.

In some aspects, the connector comprises the magnet, and the magnet is disposed below the slot. In some aspects, the connector comprises the magnet, and the magnet is disposed proximate to the connector electrical contact. In some aspects, the magnet is aligned with the connector electrical contact. In some aspects, the magnet is offset from the connector electrical contact. In some aspects, the connector comprises a plurality of connector electrical contacts, and the magnet is disposed between the plurality of connector electrical contacts. In some aspects, the connector comprises a plurality of magnets and a plurality of connector electrical contacts, and the plurality of magnets is disposed proximate to the plurality of connector electrical contacts. In some aspects, the proximal portion of the flexible elongate member comprises a first section with a first diameter and a second section with a second diameter less than the first diameter, and the connector comprises: the magnet; and a locking feature configured to engage the second diameter of the second section, and the magnet is disposed proximate to locking feature. In some aspects, the magnet is disposed proximal of the locking feature. In some aspects, the connector comprises the magnet, and the proximal portion of the flexible elongate member comprises a further magnet, and the magnet and further magnet are arranged such that opposite polarities of the magnet and further magnet attract one another. In some aspects, the further magnet is disposed proximate to the guidewire electrical contact. In some aspects, the further magnet is aligned with the guidewire electrical contact. In some aspects, the further magnet is offset from the guidewire electrical contact. In some aspects, the proximal portion of the flexible elongate member terminates at a proximal end, and the further magnet is proximate to the proximal end.

In an exemplary aspect, a system is provided. The system includes an intravascular guidewire comprising: a flexible elongate member configured to be positioned within a blood vessel of a patient, wherein the flexible elongate member comprises a proximal portion and a distal portion; at least one of a pressure sensor or a flow sensor disposed at the distal portion of the flexible elongate member and configured to obtain at least one of pressure data or flow data related to the blood vessel while the flexible elongate member is positioned within the blood vessel; a guidewire electrical contact disposed at the proximal portion of the flexible elongate member, wherein the guidewire electrical contact is in electrical communication with at least one of the pressure sensor or the flow sensor; a connector configured to be removably coupled to the intravascular guidewire, wherein the connector comprises: a slot configured to receive the proximal portion of the flexible elongate member; and a connector electrical contact configured to contact the guidewire electrical contact to establish electrical communication with at least one of the pressure sensor or the flow sensor when the proximal portion of the flexible elongate member is received within the slot, wherein at least one of: the proximal portion of the guidewire comprises a magnet proximate to the guidewire electrical contact; or the connector comprises a further magnet proximate to the connector electrical contact, wherein at least one of the magnet or the further magnet is configured to facilitate aligning the guidewire electrical contact and the connector electrical contact.

In an exemplary aspect, a system is provided. The system includes an intravascular guidewire comprising: a flexible elongate member configured to be positioned within a blood vessel of a patient, wherein the flexible elongate member comprises a proximal portion and a distal portion, wherein the proximal portion comprises a first section with a first diameter and a second section with a second diameter less than the first diameter; at least one of a pressure sensor or a flow sensor disposed at the distal portion of the flexible elongate member and configured to obtain at least one of pressure data or flow data related to the blood vessel while the flexible elongate member is positioned within the blood vessel; a guidewire electrical contact disposed at the proximal portion of the flexible elongate member, wherein the guidewire electrical contact is in electrical communication with at least one of the pressure sensor or the flow sensor; a connector configured to be removably coupled to the intravascular guidewire, wherein the connector comprises: a slot configured to receive the proximal portion of the flexible elongate member; a connector electrical contact configured to contact the guidewire electrical contact to establish electrical communication with at least one of the pressure sensor or the flow sensor when the proximal portion of the flexible elongate member is received within the slot; and a locking feature configured to engage the second diameter of the second section, wherein at least one of: the proximal portion of the guidewire comprises a magnet; or the connector comprises a further magnet proximate to the locking feature, wherein at least one of the magnet or the further magnet is configured to facilitate positioning the proximal portion of the flexible elongate member within the slot.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of aspects of the present disclosure is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 is a diagrammatic perspective view of an intravascular system, according to aspects of the present disclosure.

FIG. 2 is a diagrammatic side view of an intravascular device of the intravascular system of FIG. 1, according to aspects of the present disclosure.

FIG. 3 is a diagrammatic side view of a proximal connection portion of an intravascular device, according to aspects of the present disclosure.

FIG. 4 is a diagrammatic side view of a proximal connection portion and locking features of an intravascular device, according to aspects of the present disclosure.

FIG. 5 is a diagrammatic cross-sectional view of a proximal connection portion and locking features of an intravascular device, according to aspects of the present disclosure.

FIG. 6 is a diagrammatic top view of an intravascular device, according to aspects of the present disclosure.

FIG. 7 is a diagrammatic side view of an intravascular sensing system that includes an intravascular device, according to aspects of the present disclosure.

FIG. 8 is a diagrammatic perspective top view of an intravascular system showing a connector in an open position according to the present disclosure.

FIG. 9 is a diagrammatic top cross-sectional view of a connector, according to aspects of the present disclosure.

FIG. 10 is a diagrammatic cross-sectional view of the connection portion, the locking section, and the knob or retention section of the flexible elongate member, according to aspects of the present disclosure.

FIG. 11 illustrates a cross-sectional view of the connection portion of the flexible elongate member of FIG. 10, as seen along the lines of the section A-A taken therein, according to aspects of the present disclosure.

FIG. 12A is a diagrammatic cross-sectional view of the connection portion, the locking section, and the knob or retention section of the flexible elongate member, according to aspects of the present disclosure.

FIG. 12B is a diagrammatic cross-sectional view of the connection portion, the locking section, and the knob or retention section of the flexible elongate member, according to aspects of the present disclosure.

FIG. 13 illustrates a cross-sectional view of the connection portion of flexible elongate member of FIG. 12A and/or FIG. 12B, as seen along the lines of the section B-B taken therein, according to aspects of the present disclosure.

FIG. 14 is a diagrammatic top view of a connector of the intravascular system, while the connector is in an open position, according to aspects of the present disclosure.

FIG. 15 is diagrammatic side cross-sectional view of a connector, according to aspects of the present disclosure.

FIG. 16 is a diagrammatic cross-sectional view of the connection portion, the locking section, and the knob or retention section of the flexible elongate member, according to aspects of the present disclosure.

FIG. 17 illustrates a cross-sectional view of the connection portion of the flexible elongate member of FIG. 16, as seen along the lines of the section C-C taken therein, according to aspects of the present disclosure.

FIG. 18 is a diagrammatic top view of the connector of the intravascular system, while the connector is in an open position, according to aspects of the present disclosure.

FIG. 19 is diagrammatic cross-sectional side view of connector, according to aspects of the present disclosure.

FIG. 20 is a diagrammatic cross-sectional view of the connection portion, the locking section, and the knob or retention section of the flexible elongate member, according to aspects of the present disclosure.

FIG. 21 illustrates a cross-sectional view of the knob or retention section of the flexible elongate member of FIG. 20, as seen along the lines of the section D-D taken therein, according to aspects of the present disclosure.

FIG. 22 is a diagrammatic cross-sectional view of the connection portion, the locking section, and the knob or retention section of the flexible elongate member, according to aspects of the present disclosure.

FIG. 23 illustrates a cross-sectional view of the knob or retention section of the flexible elongate member of FIG. 22, as seen along the lines of the section E-E taken therein, according to aspects of the present disclosure.

FIG. 24 is a diagrammatic top view of the connector of the intravascular system, while the connector is in an open position, according to aspects of the present disclosure.

FIG. 25 is diagrammatic cross-sectional side view of connector, according to aspects of the present disclosure.

FIG. 26 is a diagrammatic enlarged view of a portion of the connector in FIG. 25, according to aspects of the present disclosure.

FIG. 27 is a diagrammatic top view of a locking clip, according to aspects of the present disclosure.

FIG. 28 is a diagrammatic proximal view of a locking clip, according to aspects of the present disclosure.

FIG. 29 is a diagrammatic cross-sectional view of the connection portion, the locking section, and the knob or retention section of the flexible elongate member, according to aspects of the present disclosure.

FIG. 30 illustrates a cross-sectional view of the connection portion of the flexible elongate member of FIG. 29, as seen along the lines of the section F-F taken therein, according to aspects of the present disclosure.

### DETAILED DESCRIPTION

Disclosed is an intraluminal device that advantageously utilizes magnetic alignment to properly position a proximal end of the sensing guidewire in a connector. One or more magnets are positioned at strategic locations in the connection portion of the flexible elongate member of the sensing guidewire and/or the connector. The strategic location of the magnets pull the flexible elongate member down into the recess of the connector and positions the flexible elongate member such that the locking section is aligned within the slot and the conductive portions are longitudinally aligned with the split, open-comb electrical contacts of the connector. Performing alignment utilizes such strategically placed magnets and provides correct usage of the locking core feature within the connector and thus, reduces the risk of misconnection and damage to the proximal end of the flexible elongate member.

These descriptions are provided for exemplary purposes only and should not be considered to limit the scope of present disclosure. Certain features may be added, removed, or modified without departing from the spirit of the claimed subject matter.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that may be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, intravascular catheters and intravascular guidewires. In that regard, intravascular catheters may or may not include a lumen extending along its length for receiving and/or guiding other instruments. If the intravascular catheter includes a lumen, the lumen may be centered or offset with respect to the cross-sectional profile of the device.

In most embodiments, the flexible elongate members of the present disclosure include one or more electronic, optical, or electro-optical components. For example, without limitation, a flexible elongate member may include one or more of the following types of components: a pressure sensor, a flow sensor, a temperature sensor, an imaging element, an optical fiber, an ultrasound transducer, a reflector, a mirror, a prism, an ablation element, a radio frequency (RF) electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non-medical applications. However, some embodiments of the present disclosure are particularly suited for use in the context of human vasculature. Imaging of the intravascular space, particularly the interior walls of human vasculature may be accomplished by a number of different techniques, including ultrasound (often referred to as intravascular ultrasound (“IVUS”) and intracardiac echocardiography (“ICE”)) and optical coherence tomography (“OCT”). In other instances, infrared, thermal, or other imaging modalities are utilized. Further, in some instances the flexible elongate member includes multiple electronic, optical, and/or electro-optical components (e.g., pressure sensors, temperature sensors, imaging elements, optical fibers, ultrasound transducers, reflectors, mirrors, prisms, ablation elements, RF electrodes, conductors, etc.).

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As flexible elongate members may be solid, some embodiments of the present disclosure will include a housing portion at the distal portion for receiving the electronic components. Such housing portions may be tubular structures attached to the distal portion of the elongate member. Some flexible elongate members are tubular and have one or more lumens in which the electronic components may be positioned within the distal portion.

The electronic, optical, and/or electro-optical components and the associated communication lines are sized and shaped to allow for the diameter of the flexible elongate member to be very small. For example, the outside diameter of the elongate member, such as a guidewire, catheter, or guidewire catheter, containing one or more electronic, optical, and/or electro-optical components as described herein are between about 0.0007″ (0.0178 mm) and about 0.118″ (3.0 mm), with some particular embodiments having outer diameters of approximately 0.014″ (0.3556 mm) and approximately 0.018″ (0.4572 mm)). As such, the flexible elongate members incorporating the electronic, optical, and/or electro-optical component(s) of the present application are suitable for use in a wide variety of lumens within a human patient besides those that are part or immediately surround the heart, including veins and arteries of the extremities, renal arteries, blood vessels in and around the brain, and other lumens.

“Connected” and variations thereof as used herein includes direct connections, such as being glued or otherwise fastened directly to, on, within, etc. another element, as well as indirect connections where one or more elements are disposed between the connected elements.

“Secured” and variations thereof as used herein includes methods by which an element is directly secured to another element, such as being glued or otherwise fastened directly to, on, within, etc. another element, as well as indirect techniques of securing two elements together where one or more elements are disposed between the secured elements.

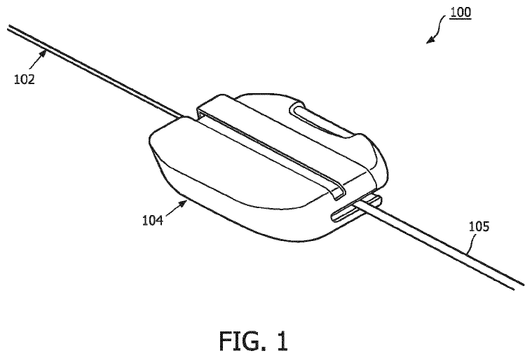

Referring initially to FIG. 1, shown therein is an intravascular system 100 according to an embodiment of the present disclosure. In that regard, the intravascular system includes an intravascular device 102 and a connector 104. As will be discussed in greater detail below, a communication cable 105 extends from the connector 104 in a direction coaxial with or parallel to the longitudinal axis of the intravascular device 102. As a result of the communication cable 105 extending coaxial with or parallel to the intravascular device, the connector 104 and communication cable 105 are less likely to catch on a patient, patient's clothing, medical equipment (including tubes, catheters, wires, leads, etc.) and/or other structures in the procedure room when maneuvering the intravascular device 102.

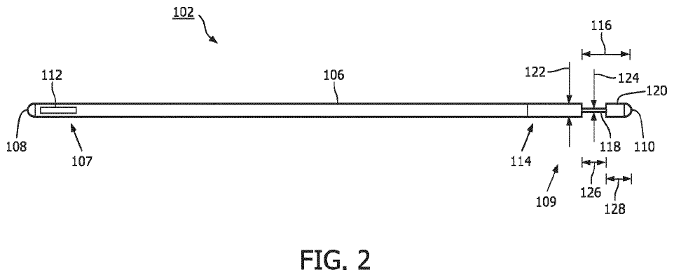

Referring now to FIG. 2, a side view of the intravascular device 102 is provided according to an embodiment of the present disclosure. As shown, the intravascular device 102 includes a flexible elongate member 106 having a distal portion 107 adjacent a distal end 108 and a proximal portion 109 adjacent a proximal end 110. A sensor 112 is positioned within the distal portion 107 of the flexible elongate member 106 proximal of the distal tip 108. Generally, the sensor 112 is representative of one or more electronic, optical, or electro-optical components. In that regard, the sensor 112 may include a pressure sensor, a flow sensor, a temperature sensor, an imaging element, an optical fiber, an ultrasound transducer, a reflector, a mirror, a prism, an ablation element, an RF electrode, a conductor, and/or combinations thereof. The specific type of component or combination of components may be selected based on an intended use of the intravascular device. In some instances, the sensor 112 is positioned less than 10 cm, less than 5, or less than 3 cm from the distal tip 108. In some instances, the sensor 112 is positioned within a housing of the intravascular device 102. In that regard, the housing may be a separate component secured to the flexible elongate member 106 in some instances. In other instances, the housing may be integrally formed as a part of the flexible elongate member 106.

The intravascular device 102 also includes a connection portion 114 adjacent the proximal portion 109 of the device. In that regard, the connection portion 114 may be spaced from the proximal end 110 of the flexible elongate member 106 by a distance 116. Generally, the distance 116 is between 0% and 50% of the total length of the flexible elongate member 106. While the total length of the flexible elongate member may be any length, in some embodiments the total length is between about 1300 mm and about 4000 mm, with some specific embodiments having a length of 1400 mm, 1200 mm, and 3000 mm. In some instances the connection portion 114 is spaced from the proximal end 110 between about 0 mm and about 1400 mm. In some specific embodiments, the connection portion 114 is spaced from the proximal end by a distance of 0 mm, 300 mm, and 1400 mm. Accordingly, in some instances the connection portion 114 is positioned at the proximal end 110. In some such embodiments, one or more aspects of the engagement and alignment features of the intravascular device 102 discussed below are positioned distal of the of the connection portion 114 instead of proximal of the connection portion 114 as shown in the embodiment of FIG. 2, or the engagement and alignment features may be omitted entirely.

In the illustrated embodiment of FIG. 2 the intravascular device 102 includes a locking section 118 extending proximally from the connection portion 114 to knob or retention section 120 that extends to proximal end 110. In the illustrated embodiment, the knob or retention section 120 is rounded to proximal end 110. In other embodiments, the knob or retention section 120 has a tapered, arcuate, and/or other changing profile as it extends proximally to proximal end 110. In that regard, in some instances the outer profile and/or diameter of the knob or retention section 120 reduces as it extends proximally to proximal end 110 such that the reduced profile and/or diameter of the proximal end facilitates easier introduction of one or more other instruments over the intravascular device. In other embodiments, the knob or retention section 120 has a constant profile as it extends proximally to proximal end 110. As knob or retention section 120 is proximal to the locking section 118, it is sometimes referred to as the proximal section.

As shown, the connection portion 114 has a diameter 122 (or other similar measurement for outer cross-section profiles for non-circular cross-sectional embodiments) while locking section 118 has a diameter 124 (again, or other similar measurement for outer cross-section profiles for non-circular cross-sectional embodiments). The diameter 124 of locking section 118 is different than the diameter 122 of connection portion 114. In that regard, the different sizes of the diameters 122, 124 create a structure that is configured to facilitate alignment and/or connection of the intravascular device 102 to a connector, such as connector 104. In the illustrated embodiment, the diameter 124 of locking section 118 is less than the diameter 122 of the connection portion 114. In some embodiments, the diameter 124 of locking section 118 is between about 40% and about 80% of diameter 122, with some particular embodiments being about 42%, 64%, and/or other percentage of diameter 122. In that regard, in some embodiments the diameter 122 of connection portion 114 is between about 0.0178 mm and about 3.0 mm, with some particular embodiments being 0.3556 mm (0.014″), 0.4572 mm (0.018″) and 0.889 mm (0.035″). Accordingly, in some embodiments the diameter 124 of locking section 118 is between about 0.007 mm and about 2.4 mm, with some particular embodiments being 0.186 mm (0.0073″), 0.23 mm, and 0.50 mm. In the illustrated embodiment, the knob or retention section 120 has a diameter that is approximately equal to diameter 122 and, therefore, greater than diameter 124. However, in other embodiments, the knob or retention section 120 has a diameter that is greater than diameter 122, less than diameter 122, greater than diameter 124, equal to diameter 124, and/or less than diameter 124. In some embodiments, locking section 118 is a section of a core wire extending through the connection portion 114. Locking section 118 and the knob or retention section 120 together may sometimes to be referred to as the locking feature.

As shown in FIG. 2, the locking section 118 extends proximally from connection portion 114 a distance 126, while the knob or retention section 120 extends proximally from locking section 118 to proximal end 110 a distance 128. Together, distances 126 and 128 equal the distance 116 that the connection portion 114 is spaced from the proximal end 110 of the intravascular device 102. In some instances, the distance 126 is between about 0.508 mm (0.020″) and about 2.54 mm (0.10″), with some particular embodiments being 0.762 mm (0.030″), 1.016 mm (0.040″), and 1.524 mm (0.060″). Further, while the transition between connection portion 114 and the locking section 118 and the transition between the locking section 118 and the knob or retention section 120 are shown as being stepped in the illustrated embodiments, in other embodiments the transitions are tapered and/or otherwise make a gradual change in outer diameter along the length of the intravascular device. In some embodiments, use of tapered and/or gradual transitions results in the proximal portion of the intravascular device 102 not having any sharp edges. In some implementations, the use of tapered and/or gradual transitions for one or both of the transitions between locking section 118 and either the connection portion 114 or the knob or retention section 120 makes cleaning the proximal portion of the device (e.g., to remove any liquids or other unwanted materials on the surface of the proximal portion of the intravascular device) easier. In some embodiments, the intravascular system 100 may include one or more features described in U.S. patent application Ser. No. 15/374,312, titled “SIDE-LOADING CONNECTORS FOR USE WITH INTRAVASCULAR DEVICES AND ASSOCIATED SYSTEMS AND METHODS,” filed Dec. 9, 2016, which is hereby incorporated by reference in its entirety.

The connection portion 114 is configured to facilitate communication between the intravascular device 102 and another device. More specifically, in some embodiments the connection portion 114 is configured to facilitate communication of data obtained by the sensor 112 to another device, such as a computing device or processor. Accordingly, in some embodiments, the connection portion 114 includes one or more conductive portions. In some implementations, the connection portion 114 may include conductive bands, rings, coatings, coils, etc. In some instances, the connection portion 114 includes one or more electrical connectors, or conductive portions, as described in U.S. patent application Ser. No. 13/931,052, titled “INTRAVASCULAR DEVICES, SYSTEMS, AND METHODS,” filed Jun. 28, 2013, which is hereby incorporated by reference in its entirety. In other embodiments, the connection portion 114 includes an optical connector. In such instances, the connection portion 114 provides an optical connection to one or more optical communication pathways (e.g., fiber optic cable) that extend along the length of the flexible elongate member 106 and are optically coupled to the sensor 112. Further, in some embodiments the connection portion 114 provides both electrical and optical connections to both electrical conductor(s) and optical communication pathway(s) coupled to the sensor 112. In that regard, it should again be noted that sensor 112 may be comprised of a plurality of elements in some instances. In some instances, the connection portion 114 may be configured to provide a physical connection to another device, either directly or indirectly. In other instances, the connection portion 114 may be configured to facilitate wireless communication between the intravascular device 102 and another device. Generally, any current or future developed wireless protocol(s) may be utilized. In yet other instances, the connection portion 114 facilitates both physical and wireless connection to another device.

As noted above, in some instances the connection portion 114 provides a connection between the sensor 112 of the intravascular device 102 and an external device. Accordingly, in some embodiments one or more electrical conductors, one or more optical pathways, and/or combinations thereof extend along the length of the flexible elongate member 106 between the connection portion 114 and the sensor 112 to facilitate communication between the connection portion 114 and the sensor 112. Generally, any number of electrical conductors, optical pathways, and/or combinations thereof may extend along the length of the flexible elongate member 106 between the connection portion 114 and the sensor 112. In some instances, between one and ten electrical conductors (or conductive portions) and/or optical pathways extend along the length of the flexible elongate member 106 between the connection portion 114 and the sensor 112. For the sake of clarity and simplicity, the embodiments of the present disclosure described below include three electrical conductors and, therefore, the connection portion 114 is described as having three separate conductive portions corresponding to the three electrical conductors.

In some embodiments, the flexible elongate member 106 includes multiple core wires. For example, the flexible elongate member 106 may include a proximal core wire (or proximal core) and a distal core wire (or distal core) that are attached to one another. The components associated with the proximal portion of the intravascular device 102 (e.g., including the proximal core wire) may be referred to a proximal subassembly, and the components associated with the distal portion of the intravascular device 102 (e.g., including the distal core wire) may be referred to a distal subassembly. The flexible elongate member may refer to one or more components of the proximal subassembly and/or the distal subassembly. In some embodiments, the flexible elongate member 106 includes features as described in U.S. patent application Ser. No. 13/931,052, titled “INTRAVASCULAR DEVICE, SYSTEMS, AND METHODS” and filed Jun. 28, 2013, which is hereby incorporated by reference in its entirety.

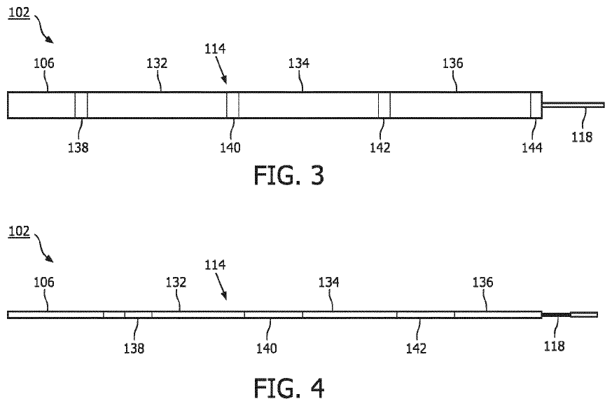

For example, as shown in FIG. 3, in some instances the connection portion 114 of the intravascular device 102 includes conductive portions 132, 134, and 136 that are separated from one another and the main body of the flexible elongate member 106 by insulating portions 138, 140, 142, and 144, with insulative portion 144 adjacent to the locking section 118. In that regard, the conductive portions 132, 134, and 136 are formed of a conductive material and are portions of a hypotube, a coil, conductive ink, conductive coating formed over a tubular member, and/or combinations thereof in some instances. In some embodiments, the conductive portions 132, 134, and 136 include features as described in U.S. patent application Ser. No. 14/143,304, titled “INTRAVASCULAR DEVICES, SYSTEMS, AND METHODS” and filed Dec. 30, 2013, which is hereby incorporated by reference in its entirety. It is understood that the total number of communication pathways and/or the number of electrical conductors and/or optical pathways is different in other embodiments and, therefore, the number of conductive portions (or optical connectors) included in connection portion is different as well. More specifically, the number of communication pathways and the number of electrical conductors and optical pathways extending along the length of the flexible elongate member 106 may be selected based on the desired functionality of the sensor 112 and the corresponding elements that define sensor 112 to provide such functionality. As a result, the number and type of connections provided by connection portion 114 are likewise determined by the desired functionality of the sensor 112, the corresponding elements that define sensor 112 to provide such functionality, and the communication needs for such elements. Further still, in some instances, one or more of the insulating portions 138, 140, 142, and 144 is omitted. For example, as shown in the exemplary embodiment of FIG. 4, insulating portion 144 has been omitted.

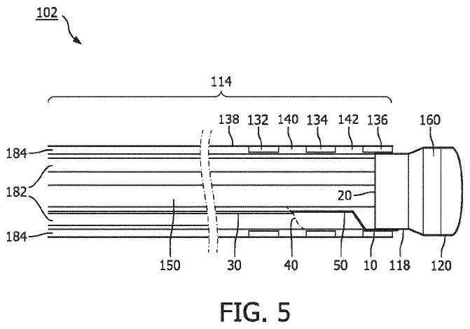

Referring now to FIG. 5, shown therein is a diagrammatic cross-sectional view of the connection portion 114, the locking section 118 and the knob or retention section 120 of the intravascular device 102, according to aspects of the present disclosure. In some embodiments, the connection portion 114 includes three conductive portions 132, 134, and 136 that are separated from one another and the main body of the flexible elongate member by insulating portions 138, 140, and 142. As the conductive portions 132, 134, and 136 and the insulating portions 138, 140 and 142 are in annular-ring shapes around a circumference of the flexible elongate member, cross sections of them appear on either side of the connection portion 114. One, a plurality, or all of the conductive portions 132, 134, and 136 is electrically coupled to respective conductor or conductive element (e.g., a conductive wire 30, 40, 50). In the example shown in FIG. 5, conductive portion 132 is electrically coupled to conductive wire 30, conductive portion 134 is electrically coupled to conductive wire 40, and conductive portion 136 is electrically coupled to conductive wire 50. In some instances, the conductive wires 30, 40 and 50 are positioned within an open space 182 defined by a hypotube 184 that forms part of the flexible elongate member. Conductive wires 30, 40 and 50 are electrically coupled to sensor and extend proximally through the flexible elongate member. In some embodiments, the flexible elongate member includes a metal core 150 that extends through the connection portion 114. In some implementations, locking section 118 and the knob or retention section 120 are parts of an integral component referred to as a locking core 160. Locking core 160 is separate from the metal core 150 and is attached by soldering to the proximal end of the connection portion 114 at interface 10 with conductive portion 136 and at interface 20 with a proximal end of the metal core 150.

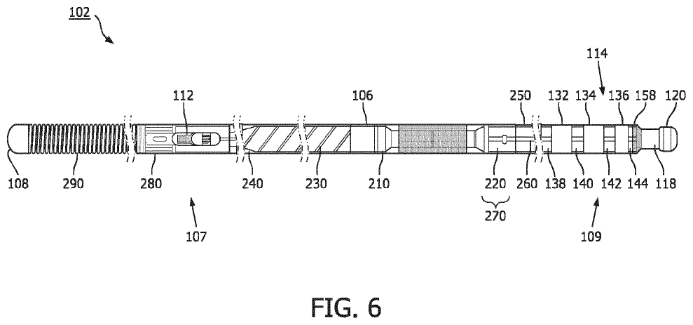

Referring now to FIG. 6, shown there is a diagrammatic top view of intravascular device 102, according to aspects of the present disclosure. The intravascular device 102 may be an intravascular guidewire sized and shaped for positioning within a vessel of a patient. The intravascular device 102 may include the sensor 112. For example, the sensor 112 may be a pressure sensor configured to measure a pressure of blood flow within the vessel of the patient. The intravascular device 102 includes the flexible elongate member 106. The sensor 112 is disposed at the distal portion 107 of the flexible elongate member 106. The sensor 112 may be mounted at the distal portion 107 within a housing 280 in some embodiments. A flexible tip coil 290 extends between the housing 280 and the distal end 108. The connection portion 114 is disposed at the proximal portion 109 of the flexible elongate member 106. The connection portion includes the conductive portions 132, 134, 136 that are separated from one another and the main body of the flexible elongate member 106 by insulating portions 138, 140, 142, and 144. In some embodiments, the conductive portions 132, 134, 136 may be conductive ink that is printed and/or deposited around the flexible elongate member. In some embodiments, the conductive portions 132, 134, 136 are conductive, metallic rings that are positioned around the flexible elongate member. The locking section 118 and the knob or retention section 120 are disposed at the proximal portion 109 of the flexible elongate member 106.

The intravascular device 102 in FIG. 6 includes a distal core 210 and a proximal core 220. The distal core 210 and the proximal core 220 are metallic components forming part of the body of the intravascular device 102. For example, the distal core 210 and the proximal core 220 are flexible metallic rods that provide structure for the flexible elongate member 106. The diameter of the distal core 210 and the proximal core 220 may vary along its length.

In some embodiments, the intravascular device 102 comprises a distal assembly and a proximal assembly that are electrically and mechanically joined together, which results in electrical communication between the sensor 112 and the conductive portions 132, 134, 136. For example, pressure data obtained by the sensor 112 (in this example, sensor 112 is a pressure sensor) may be transmitted to the conductive portions 132, 134, 136. Control signals from a computer in communication with the intravascular device 102 may be transmitted to the sensor 112 via the conductive portions 132, 134, 136. The distal subassembly may include the distal core 210. The distal subassembly may also include the sensor 112, the conductive members 230, and/or one or more layers of polymer/plastic 240 surrounding the conductive members 230 and the core 210. For example, the polymer/plastic layer(s) may protect the conductive members 230. The proximal subassembly may include the proximal core 220. The proximal subassembly may also include one or more layers of polymer layer(s) 250 (hereinafter polymer layer 250) surrounding the proximal core 220 and/or conductive ribbons 260 embedded within the one or more layers of polymer layer(s) 250. In some embodiments, the proximal subassembly and the distal subassembly may be separately manufactured. During the assembly process for the intravascular device 102, the proximal subassembly and the distal subassembly may be electrically and mechanically joined together. As used herein, flexible elongate member may refer to one or more components along the entire length of the intravascular device 102, one or more components of the proximal subassembly (e.g., including the proximal core 220, etc.), and/or one or more components the distal subassembly 210 (e.g., including the distal core 210, etc.).

In various embodiments, the intravascular device 102 may include one, two, three, or more core wires extending along its length. For example, a single core wire may extend substantially along the entire length of the flexible elongate member 106. In such embodiments, a locking section 118 and the knob or retention section 120 may be integrally formed at the proximal portion of the single core wire. The sensor 112 may be secured at the distal portion of the single core wire. In other embodiments, such as the embodiment illustrated in FIG. 6, the locking section 118 and the knob or retention section 120 may be integrally formed at the proximal portion of the proximal core 220. The sensor 112 may be secured at the distal portion of the distal core 210. The intravascular device 102 includes one or more conductive members 230 in communication with the sensor 112. For example, the conductive members 230 may be one or more electrical wires that are directly in communication with the sensor 112. In some instances, the conductive members 230 are electrically and mechanically coupled to the sensor 112 by, e.g., soldering. In some instances, the conductive members 230 comprise two or three electrical wires (e.g., a bifilar cable or a trifilar cable). An individual electrical wire may include a bare metallic conductor surrounded by one or more insulating layers. The conductive members 230 may extend along the length of the distal core 210. For example, at least a portion of the conductive members 230 may be spirally wrapped around the distal core 210.

The intravascular device 102 includes one or more conductive ribbons 260 at the proximal portion of the flexible elongate member 106. The conductive ribbons 260 are embedded within polymer layer(s) 250. The conductive ribbons 260 are directly in communication with the conductive portions 132, 134, and/or 136. In some instances, the conductive members 230 are electrically and mechanically coupled to the sensor 112 by, e.g., soldering. In some instances, the conductive portions 132, 134, and/or 136 comprise conductive ink (e.g., metallic nano-ink, such as silver or gold nano-ink) that is deposited or printed directed over the conductive ribbons 260.

As described herein, electrical communication between the conductive members 230 and the conductive ribbons 260 may be established at the connection region 270 of the flexible elongate member 106. By establishing electrical communication between the conductive members 230 and the conductive ribbons 260, the conductive portions 132, 134, 136 may be in electrically communication with the sensor 112.

In some embodiments represented by FIG. 6, intravascular device 102 includes a locking section 118 and the knob or retention section 120. Different from the locking core 160 (including the locking section 118 and the knob or retention section 120) in FIG. 5, which is soldered to the metal core, locking section 118 and section 120 in FIG. 6 are integral with proximal core 220. To form locking section 118, a machining process is necessary to remove polymer layer 250 and conductive ribbons 260 in locking section 118 and to shape proximal core 220 in locking section 118 to the desired shape. As shown in FIG. 6, locking section 118 includes a reduced diameter while the knob or retention section 120 has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons.

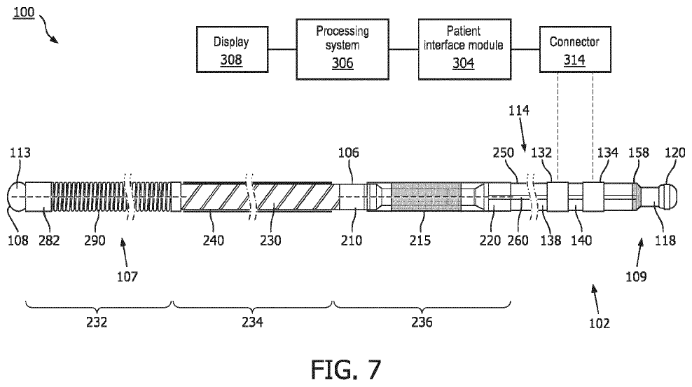

FIG. 7 is a diagrammatic side view of an intraluminal (e.g., intravascular) sensing system 100 that includes an intravascular device 102 comprising conductive members 230 (e.g., a multi-filar electrical conductor bundle) and conductive ribbons 260, according to aspects of the present disclosure. The intravascular device 102 may be an intravascular guidewire sized and shaped for positioning within a blood vessel of a patient. The intravascular device 102 includes a distal tip 108 and a sensor 113. For example, the sensor 113 may be a pressure sensor and/or flow sensor configured to measure a pressure of blood flow within the vessel of the patient, or another type of sensor including but not limited to a temperature or imaging sensor, or combination sensor measuring more than one property. For example, the flow data obtained by a flow sensor may be used to calculate physiological variables such as coronary flow reserve (CFR). The intravascular device 102 includes a flexible elongate member 106. The sensor 113 is disposed at a distal portion 107 of the flexible elongate member 106. The sensor 113 may be mounted at the distal portion 107 within a housing 280 in some embodiments. A flexible tip coil 290 extends distally from the housing 282 at the distal portion 107 of the flexible elongate member 106. A connection portion 114 located at a proximal end of the flexible elongate member 106 includes conductive portions 132, 134. In some embodiments, the conductive portions 132, 134 may be conductive ink that is printed and/or deposited around the connection portion 114 of the flexible elongate member 106. In some embodiments, the conductive portions 132, 134 are conductive, may be metallic bands or rings that are positioned around the flexible elongate member. A locking area is formed by a collar or locking section 118 and knob or retention section 120 are disposed at the proximal portion 109 of the flexible elongate member 106.

The intravascular device 102 in FIG. 7 includes core wire comprising a distal core 210 and a proximal core 220. The distal core 210 and the proximal core 220 are metallic components forming part of the body of the intravascular device 102. For example, the distal core 210 and the proximal core 220 may be flexible metallic rods that provide structure for the flexible elongate member 106. The distal core 210 and/or the proximal core 220 may be made of a metal or metal alloy. For example, the distal core 210 and/or the proximal core 220 may be made of stainless steel, Nitinol, nickel-cobalt-chromium-molybdenum alloy (e.g., MP35N), and/or other suitable materials. In some embodiments, the distal core 210 and the proximal core 220 are made of the same material. In other embodiments, the distal core 210 and the proximal core 220 are made of different materials. The diameter of the distal core 210 and the proximal core 220 may vary along their respective lengths. A joint between the distal core 210 and proximal core 220 is surrounded and contained by a hypotube 215. The sensor 113 may in some cases be positioned at a distal end of the distal core 210.

In some embodiments, the intravascular device 102 comprises a distal subassembly and a proximal subassembly that are electrically and mechanically joined together, which creates an electrical communication between the sensor 113 and the conductive portions 132, 134. For example, flow data obtained by the sensor 113 (in this example, sensor 113 is a flow sensor) may be transmitted to the conductive portions 132, 134. In an exemplary embodiment, the sensor 113 is a single ultrasound transducer element. The transducer element emits ultrasound signals and receives echoes. The transducer element generates electrical signals representative of the echoes. The signal carrying filars carry this electrical signal from the sensor at the distal portion to the connector at the proximal portion. The processing system 306 processes the electrical signals to extract the flow velocity of the fluid.

Control signals from a processing system 306 (e.g., a processor circuit of the processing system 306) in communication with the intravascular device 102 may be transmitted to the sensor 113 via a connector 314 that is attached to the conductive portions 132, 134. The distal subassembly may include the distal core 210. The distal subassembly may also include the sensor 113, the conductive members 230, and/or one or more layers of insulative polymer/plastic 240 surrounding the conductive members 230 and the core 210. For example, the polymer/plastic layer(s) may insulate and protect the conductive members of the multi-filar cable or conductor bundle 230. The proximal subassembly may include the proximal core 220. The proximal subassembly may also include one or more polymer layers 250 (hereinafter polymer layer 250) surrounding the proximal core 220 and/or conductive ribbons 260 embedded within the one or more insulative and/or protective polymer layer 250. In some embodiments, the proximal subassembly and the distal subassembly are separately manufactured. During the assembly process for the intravascular device 102, the proximal subassembly and the distal subassembly may be electrically and mechanically joined together. As used herein, flexible elongate member may refer to one or more components along the entire length of the intravascular device 102, one or more components of the proximal subassembly (e.g., including the proximal core 220, etc.), and/or one or more components the distal subassembly 410 (e.g., including the distal core 210, etc.). Accordingly, flexible elongate member may refer to the combined proximal and distal subassemblies described above. The joint between the proximal core 220 and distal core 210 is surrounded by the hypotube 215.

In various embodiments, the intravascular device 102 may include one, two, three, or more core wires extending along its length. For example, a single core wire may extend substantially along the entire length of the flexible elongate member 106. In such embodiments, a locking section 118 and a section 120 may be integrally formed at the proximal portion of the single core wire. The sensor 113 may be secured at the distal portion of the single core wire. In other embodiments, such as the embodiment illustrated in FIG. 7, the locking section 118 and the section 120 may be integrally formed at the proximal portion of the proximal core 220. The sensor 113 may be secured at the distal portion of the distal core 210. The intravascular device 102 includes one or more conductive members 230 (e.g., a multi-filar conductor bundle or cable) in communication with the sensor 113. For example, the conductive members 230 may be one or more electrical wires that are directly in communication with the sensor 113. In some instances, the conductive members 230 are electrically and mechanically coupled to the sensor 113 by, e.g., soldering. In some instances, the conductor bundle 230 comprises two or three electrical wires (e.g., a bifilar cable or a trifilar cable). An individual electrical wire may include a bare metallic conductor surrounded by one or more insulating layers. The conductive members 230 may extend along the length of the distal core 210. For example, at least a portion of the conductive members 230 may be spirally wrapped around the distal core 210, minimizing or eliminating whipping of the distal core within tortuous anatomy.

The intravascular device 102 includes one or more conductive ribbons 260 at the proximal portion of the flexible elongate member 106. The conductive ribbons 260 are embedded within polymer layer 250. The conductive ribbons 260 are directly in communication with the conductive portions 132 and/or 134. In some instances, a multi-filar conductor bundle 230 is electrically and mechanically coupled to the sensor 113 by, e.g., soldering. In some instances, the conductive portions 132 and/or 134 comprise conductive ink (e.g., metallic nano-ink, such as copper, silver, gold, or aluminum nano-ink) that is deposited or printed directed over the conductive ribbons 260.

As described herein, electrical communication between the conductive members 230 and the conductive ribbons 260 may be established at the connection portion 114 of the flexible elongate member 106. By establishing electrical communication between the conductor bundle 230 and the conductive ribbons 260, the conductive portions 132, 134 may be in electrical communication with the sensor 113.

In some embodiments represented by FIG. 7, the intravascular device 102 includes a locking section 118 and knob or retention section 120. To form locking section 118, a machining process is used to remove polymer layer 250 and conductive ribbons 260 in locking section 118 and to shape proximal core 220 in locking section 118 to the desired shape. As shown in FIG. 7, locking section 118 includes a reduced diameter while knob or retention has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons 260.

In some embodiments, a connector 314 provides electrical connectivity between the conductive portions 132, 134 and a patient interface monitor 304. The Patient Interface Monitor (PIM) 304 may in some cases connect to a console or processing system 306, which includes or is in communication with a display 308.

The system 100 may be deployed in a catheterization laboratory having a control room. The processing system 306 may be located in the control room. Optionally, the processing system 306 may be located elsewhere, such as in the catheterization laboratory itself. The catheterization laboratory may include a sterile field while its associated control room may or may not be sterile depending on the procedure to be performed and/or on the health care facility. In some embodiments, device 102 may be controlled from a remote location such as the control room, such that an operator is not required to be in close proximity to the patient.

The intraluminal device 102, PIM 304, and display 308 may be communicatively coupled directly or indirectly to the processing system 306. These elements may be communicatively coupled to the medical processing system 306 via a wired connection such as a standard copper multi-filar conductor bundle 230. The processing system 306 may be communicatively coupled to one or more data networks, e.g., a TCP/IP-based local area network (LAN). In other embodiments, different protocols may be utilized such as Synchronous Optical Networking (SONET). In some cases, the processing system 306 may be communicatively coupled to a wide area network (WAN).

The PIM 304 transfers the received signals to the processing system 306 where the information is processed and displayed (e.g., as physiology data in graphical, symbolic, or alphanumeric form) on the display 308. The console or processing system 306 may include a processor and a memory. The processing system 306 may be operable to facilitate the features of the intravascular sensing system 100 described herein. For example, the processor may execute computer readable instructions stored on the non-transitory tangible computer readable medium.

The PIM 304 facilitates communication of signals between the processing system 306 and the intraluminal device 102. The PIM 304 may be communicatively positioned between the processing system 306 and the intraluminal device 102. In some embodiments, the PIM 304 performs preliminary processing of data prior to relaying the data to the processing system 306. In examples of such embodiments, the PIM 304 performs amplification, filtering, and/or aggregating of the data. In an embodiment, the PIM 304 also supplies high-and low-voltage DC power to support operation of the intraluminal device 102 via the conductive members 230.

A multi-filar cable or transmission line bundle 230 can include a plurality of conductors, including one, two, three, four, five, six, seven, or more conductors. In the example shown in FIG. 7, the multi-filar conductor bundle 230 includes two straight portions 232 and 236, where the multi-filar conductor bundle 230 lies parallel to a longitudinal axis of the flexible elongate member 106, and a spiral portion 234, where the multi-filar conductor bundle 230 is wrapped around the exterior of the flexible elongate member 106 and then overcoated with an insulative and/or protective polymer 240. Communication, if any, along the multi-filar conductor bundle 230 may be through numerous methods or protocols, including serial, parallel, and otherwise, where one or more filars of the bundle 230 carry signals. One or more filars of the multi-filar conductor bundle 230 may also carry direct current (DC) power, alternating current (AC) power, or serve as a ground connection.

The display or monitor 308 may be a display device such as a computer monitor or other type of screen. The display or monitor 308 may be used to display selectable prompts, instructions, and visualizations of imaging data to a user. In some embodiments, the display 308 may be used to provide a procedure-specific workflow to a user to complete an intraluminal imaging procedure.

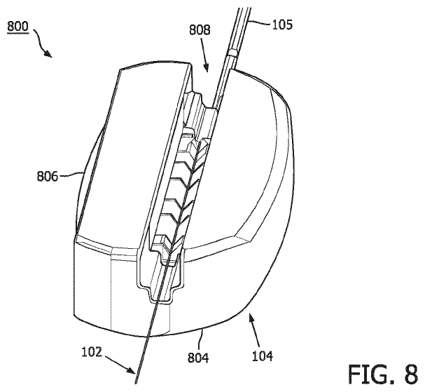

FIG. 8 is a diagrammatic perspective top view of an intravascular system showing a connector in an open position according to the present disclosure. As shown in FIG. 8, shown therein are aspects of an intravascular system 800 having an intravascular device 102 and a connector 104 according to the present disclosure. The connector 104 includes a component 804 and a component 806. The component 804 includes a recess 808 sized and shaped to receive the intravascular device 102. The component 806 is movable with respect to the component 804. In particular, the component 806 is slidable with respect to the component 804 to facilitate insertion of the intravascular device 102 into the connector 104 and subsequent engagement of the connector with the received intravascular device that results in one or more electrical connections between the intravascular device and the connector. The sliding movement of the component 806 relative to the component 804 can be parallel to a longitudinal axis of the component 804 and/or the longitudinal axis of an intravascular device received within the connector 104. The communication cable 105 extends from the connector 104 such that the communication cable 105 is coaxial with or parallel to the intravascular device 102 received within the connector 104.

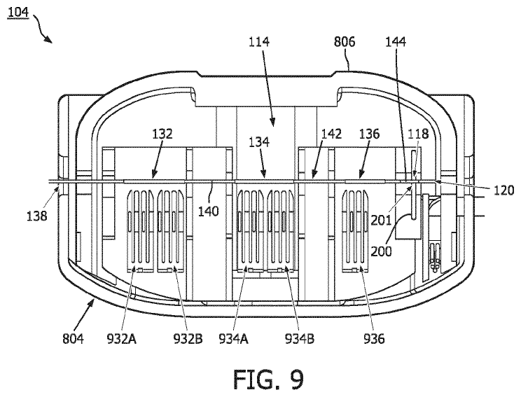

FIG. 9 shows a diagrammatic cross-sectional view of the connector 104. In some embodiments, component 806 includes split, open-comb electrical contacts 932A, 932B, 934A, 934B, and 936. Component 804 includes a locking clip 200. The locking clip 200 includes a slit 201 sized and shaped to receive locking section 118 while section 120 is proximal to the locking clip 200. When connection portion 114 is received within recess 208 and locking section 118 is received within slit 201, conductive portion 132 is aligned with electrical contacts 932A and 932B along a direction of the relative movement between component 204 and component 206. Similarly, along the same direction of movement, conductive portion 134 is aligned with electrical contacts 934A and 934B and conductive portion 136 is aligned with electrical contacts 936. In some embodiments, conductive portions 132, 134, and 136 are separated by insulating portions 138, 140, and 142. In some instances, connection portion 114 also includes an insulating portion 144 distal to locking section 118. As locking section 118 is of a reduced diameter as compared to section 120 and insulating portion 144 (or conductive portion 136 if insulating portion 144 is not present), the connection portion 114 is prevented from moving along its lengthwise direction, either distally or proximally.

Further, the open-comb electrical contacts are particularly well-suited to facilitate proper electrical connection between the connector 104 and an intravascular device 102 positioned within the recess 808 of component 804 when the component 806 is translated relative to the component 804 from the open position towards the closed position. Further still, the open-comb configuration allows for the intravascular device to be rotated with respect to the connector while maintaining a proper electrical connection. Thus, the open-comb configuration allows a user (e.g., surgeon) to keep the connector 104 connected to the intravascular device while the intravascular device is moved or advanced through the vasculature with little resistance to rotational movement of the intravascular device. In other words, the intravascular device can be moved through the vasculature, undergoing various twists and turns, without the connector 104 needing to move with the rotations of the intravascular device. Also, the open-comb configuration helps ensure good electrical contact due to the multiple fingers for each of the contacts. In addition, the open end of the open-comb configuration provides a good guide for ensuring that the intravascular device is correctly positioned when the component 806 is closed. While various advantages of the open-comb configuration have been described, it is understood that any appropriately sized electrical contacts can be utilized, including a single contact or a plurality of contacts.

Before continuing, it should be noted that the examples described above are provided for purposes of illustration and are not intended to be limiting. Other devices and/or device configurations may be utilized to carry out the operations described herein.

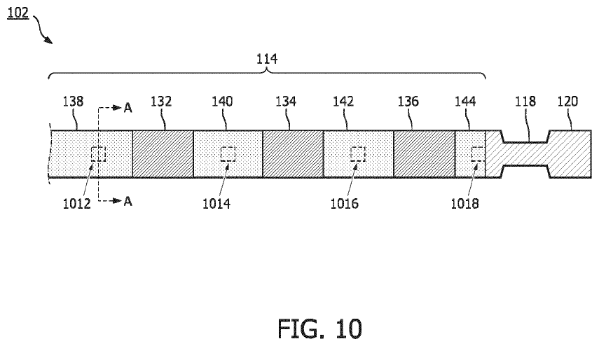

FIG. 10 is a diagrammatic cross-sectional view of the connection portion 114, the locking section 118, and the knob or retention section 120 of the flexible elongate member 106, according to aspects of the present disclosure. In some embodiments, the connection portion 114 includes three conductive portions 132, 134, and 136 that are separated from one another and the main body of the flexible elongate member by insulating portions 138, 140, 142, and 144. As the conductive portions 132, 134, and 136 and the insulating portions 138, 140, 142, and 144 are in annular-ring shapes around a circumference of the flexible elongate member, cross sections of them appear on either side of the connection portion 114. In some embodiments, one or more magnets may be embedded within a polymer layer of the connection portion 114. For example, magnets 1012 may be embedded within the polymer layer 180 in insulating portion 138, magnets 1014 may be embedded within the polymer layer 180 in insulating portion 140, magnets 1016 may be embedded within the polymer layer 180 in insulating potion 142, and magnets 1018 may be embedded within the polymer layer 180 in insulating portion 144. In some embodiments, magnets 1012, 1014, 1016, and 1018, when combined with magnets in the connector 104, described hereafter, pull the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, magnets 1012, 1014, 1016, and 1018 are attracted to metal in connector 104, pulling the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some instances, the insulating portions 138, 140, 142, and 144 can be segments or regions of the polymer layer 180 (and not separate components), such as the polymer layer in which the conductive ribbons are embedded.

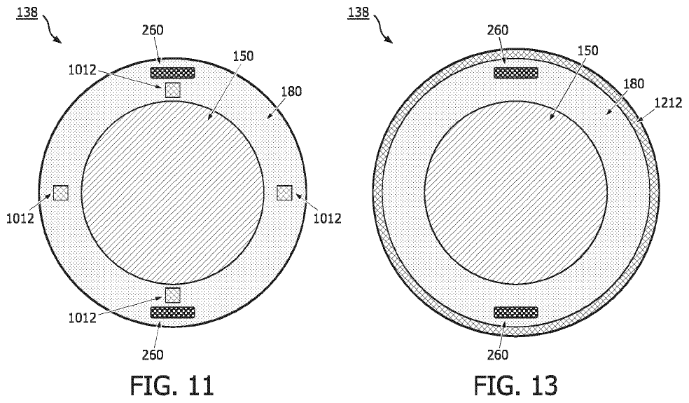

FIG. 11 illustrates a cross-sectional view of the connection portion 114 of the flexible elongate member 106 of FIG. 10, as seen along the lines of the section A-A taken therein, according to aspects of the present disclosure. In some embodiments, the connection portion 114 includes the metal core 150, the conductive ribbons 260, and the polymer layer 180 that insulates the conductive ribbons 260 from one another and also insulates conductive ribbons 260 from the metal core 150. In some embodiments, one or more magnets may be embedded within the polymer layer 180 of the connection portion 114. For example, magnets 1012 may be embedded within the polymer layer 180.

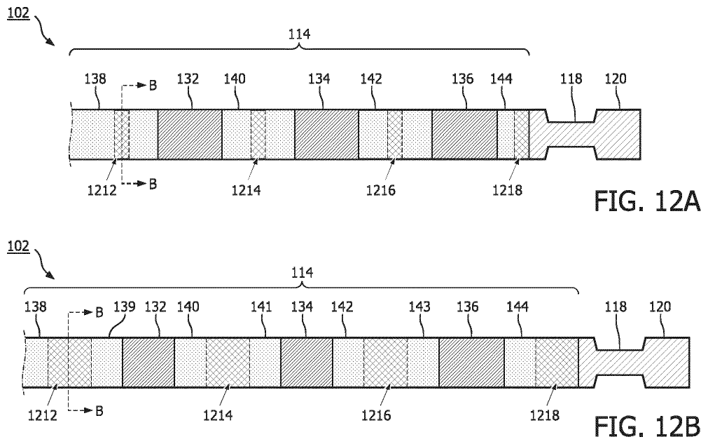

FIG. 12A and FIG. 12B are diagrammatic cross-sectional views of the connection portion 114, the locking section 118, and the knob or retention section 120 of the flexible elongate member 106, according to aspects of the present disclosure. FIG. 13 illustrates a cross-sectional view of the connection portion 114 of the flexible elongate member 106 of FIG. 12A and/or FIG. 12B, as seen along the lines of the section B-B taken therein, according to aspects of the present disclosure. FIGS. 12A, 12B, and 13 include features similar to those described in FIGS. 10 and 11. In the embodiment of FIGS. 12A, 12B, and 13, the magnets 1212, 1214, 1216, and 1218 are formed in annular-ring shapes around a circumference of the connection portion 114. In some embodiments, magnets 1212, when combined with magnets 1214, 1216, and 1218 and when combined with magnets in the connector 104, described hereafter, pull the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, magnets 1012, 1014, 1016, and 1018 are attracted to metal in connector 104, pulling the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104.

FIG. 12B is different than FIG. 12A in that the connection portion 114 in FIG. 12B is longer (e.g., larger in the longitudinal direction) than the connection portion 114 in FIG. 12A. FIG. 12B may be advantageous in situations where space is not available in the outside diameter of the intravascular device 102 for additional components such as the magnets 1212, 1214, 1216, and 1218. By extending the length of the intravascular device 102 (e.g., the connection portion 114), additional space is created in the length of the intravascular device 102 (while the outside diameter of the intravascular device 102 remains the same) for the magnets 1212, 1214, 1216, and 1218. The magnets 1212, 1214, 1216, and 1218 can be directly adjacent to the insulating portions 138-144, respectively. The insulating portions 138-144 can space from the magnets 1212, 1214, 1216, and 1218 from the conductive portions 132, 134, and 136. In some instances, the magnets 1212, 1214, 1216, and 1218 can be directly adjacent to the conductive portions 132, 134, and 136. The outer surfaces of the magnets 1212, 1214, 1216, and 1218, the outer surfaces of the conductive portions 132, 134, and 136, and the outer surfaces of the insulating portions 138-144 can be continuous with one another.

In general, while multiple magnets 1212, 1214, 1216, and 1218, the connection portion 114 can include one, two, three, four, or more magnets.

In some instances, a magnet can directly surround the core wire 150 in FIG. 13 such that the magnet directly contacts the core wire 150.

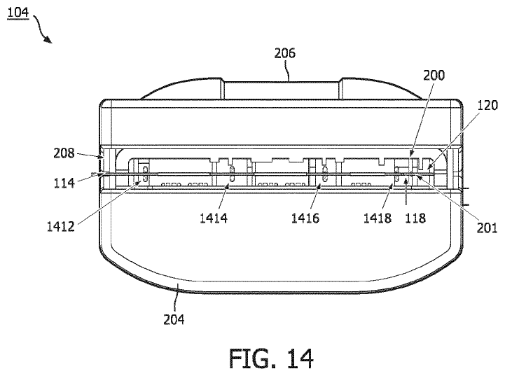

Referring now to FIG. 14, shown therein is a diagrammatic top view of the connector 104 of the intravascular system 100, while the connector is in an open position, according to aspects of the present disclosure. The example connector 104 shown in FIG. 14 includes a component 204 and a component 206. The component 204 includes a recess 208 sized and shaped to receive the connection portion 114 of the flexible elongate member 106. The component 206 is movable with respect to the component 204. In particular, the component 206 is slidable with respect to the component 204 to facilitate insertion of an intravascular device 102 into the connector 104 and subsequent engagement of the connector 104 with the received intravascular device 102 that results in one or more electrical connections between the intravascular device 102 and the connector 104. The sliding movement of the component 206 relative to the component 204 may be parallel to a longitudinal axis of the component 204 and/or the longitudinal axis of an intravascular device 102 received within the connector 104. Component 204 includes a locking clip 200. The locking clip 200 includes a slit 201 sized and shaped to receive locking section 118 while section 120 is proximal to the locking clip 200.

In some embodiments, one or more magnets may be positioned within connector 104. For example, magnets 1412, 1414, 1416, and 1418 may be positioned below the respective areas where insulating portions 138, 140, 142, and 144 of the connection portion 114 of the flexible elongate member 106 will be positioned when the intravascular device 102 is inserted into the connector 104. In some embodiments, magnets 1412, 1414, 1416, and 1418 are advantageously positioned adjacent or proximate to conductive portions 132, 134, and 136. In some embodiments, magnets 1412, 1414, 1416, and 1418 have an opposite polarity to that of magnets 1012, 1014, 1016, and 1018 in the connection portion 114 of the flexible elongate member 106. Thus, in some embodiments, a magnetic attraction between magnets 1412, 1414, 1416, and 1418 and magnets 1012, 1014, 1016, and 1018 pulls the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, magnets 1412, 1414, 1416, and 1418 attract metal in intravascular device 102, pulling the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104.

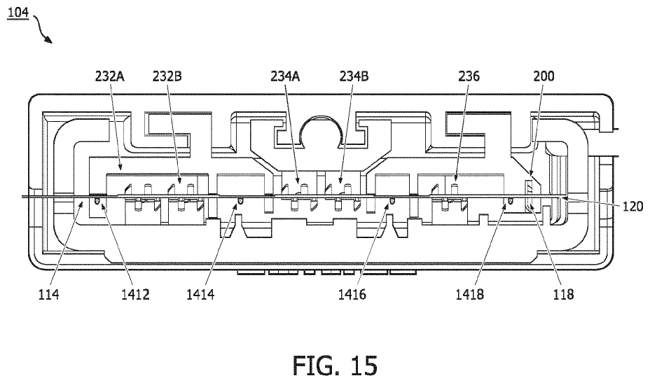

FIG. 15 is diagrammatic cross-sectional side view of connector 104, according to aspects of the present disclosure. FIG. 15 shows how conductive portions engage the split, open-comb electrical contacts 232A, 232B, 234A, 234B, and 236 and how locking section 118 is received within slit 201 of locking clip 200. In some instances, each of the electrical contacts has two arms that bend upward and two arms that bend downwards. Each of the electrical contacts can also have more or less arms bending different directions. For example, each of the electrical contacts may have one or three arms bending upwards and one or three arms bending downwards. In some embodiments, the slit 201 extends halfway through the height of the locking clip 200. Advantageously, the engagement of locking section 118 and slit 201 of locking clip 200 ensure reliable electrical connection between conductive portions 132, 134, and 136 of connection portion 114 and split, open-comb electrical contacts 232A, 232B, 234A, 234B, and 236 in connector 104. In some embodiments, magnets 1412, 1414, 1416, and 1418 have an opposite polarity to that of magnets 1012, 1014, 1016, and 1018 in the connection portion 114 of the flexible elongate member 106. Thus, in some embodiments, a magnetic attraction between magnets 1412, 1414, 1416, and 1418 and magnets 1012, 1014, 1016, and 1018 pulls the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, magnets 1412, 1414, 1416, and 1418 attract metal in intravascular device 102, pulling the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104.

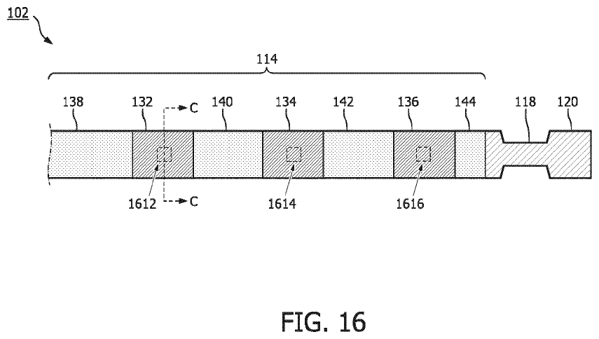

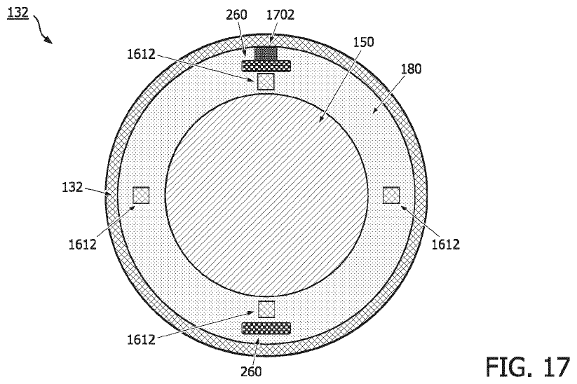

FIG. 16 is a diagrammatic cross-sectional view of the connection portion 114, the locking section 118, and the knob or retention section 120 of the flexible elongate member 106, according to aspects of the present disclosure. FIG. 17 illustrates a cross-sectional view of the connection portion 114 of the flexible elongate member 106 of FIG. 16, as seen along the lines of the section C-C taken therein, according to aspects of the present disclosure. FIGS. 16 and 17 include features similar to those described in FIGS. 10 and 11. In the embodiments of FIG. 16, the magnets 1612 may be embedded within the polymer layer 180 in conductive portion 132, magnets 1614 may be embedded within the polymer layer 180 in conductive portion 134, magnets 1616 may be embedded within the polymer layer 180 in conductive potion 146. In the embodiments of FIG. 17, magnets 1612 may be embedded within the polymer layer 180 where conductive portion 132 is electrically coupled to one of the two conductive ribbons 260 via electrical connector 1702. In some embodiments, magnets 1612, when combined with magnets 1614 and 1616 and when combined with magnets in the connector 104, described hereafter, pull the flexible elongate member 106 down into the recess 208 so flexible elongate member 106 is positioned inside recess 208 of the connector 140, locking section 118 is positioned aligned with slot 201, and conductive portions 132, 134 and, 136 are longitudinally aligned with the split, open-comb electrical contacts 932A, 932B, 934A, 934B, and 936 of connector 104. In some embodiments, magnets 1612, when combined with magnets 1614 and 1616, are attracted to metal in connector 104, pulling the flexible elongate member 106 down into the recess 208 so flexible elongate member 106 is positioned inside recess 208 of the connector 140, locking section 118 is positioned aligned with slot 201, and conductive portions 132, 134 and, 136 are longitudinally aligned with the split, open-comb electrical contacts 932A, 932B, 934A, 934B, and 936 of connector 104.

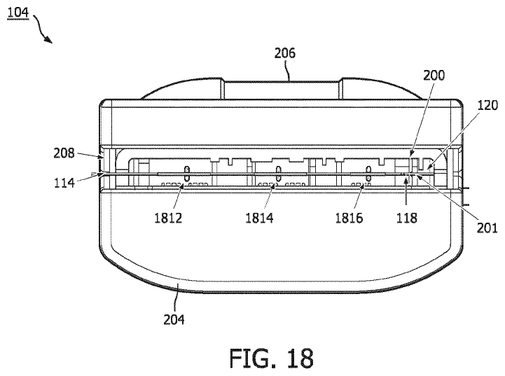

FIG. 18 is a diagrammatic top view of the connector 104 of the intravascular system 100, while the connector is in an open position, according to aspects of the present disclosure. FIG. 18 include features similar to those described in FIG. 14. In the embodiments of FIG. 18, one or more magnets may be positioned within connector 104 such that magnets 1812, 1814, and 1816 may be aligned with the split, open-comb electrical contacts 932A, 932B, 934A, 934B, and 936. Magnet 1812 may be positioned between split, open-comb electrical contacts 932A and 932B, magnet 1814 may be positioned between split, open-comb electrical contacts 934A and 934B, and magnet 1816 may be positioned offset to split, open-comb electrical contacts 936. In some embodiments, magnets 1812, 1814, and 1816 have an opposite polarity to that of magnets 1612, 1614, and 1616 in the connection portion 114 of the flexible elongate member 106. Thus, in some embodiments, a magnetic attraction between magnets 1812, 1814, and 1816 and magnets 1612, 1614, and 1616 pulls the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, magnets 1812, 1814, and 1816 attract metal in intravascular device 102, pulling the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104.

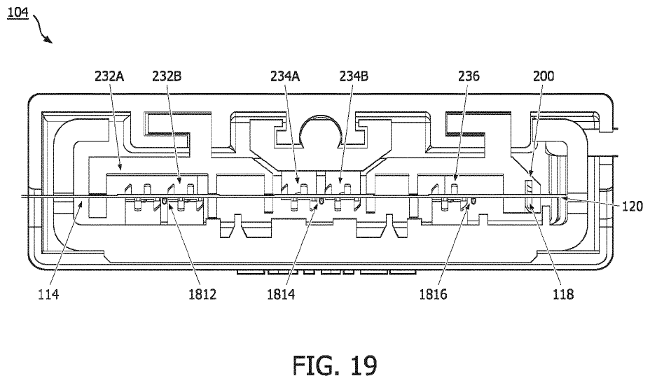

FIG. 19 is diagrammatic cross-sectional side view of connector 104, according to aspects of the present disclosure. FIG. 19 include features similar to those described in FIG. 15. In some embodiments, magnets 1812, 1814, and 1816 have an opposite polarity to that of magnets 1612, 1614, and 1616 in the connection portion 114 of the flexible elongate member 106. Thus, in some embodiments, a magnetic attraction between magnets 1812, 1814, and 1816 and magnets 1612, 1614, and 1616 pulls the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, magnets 1812, 1814, and 1816 attract metal in the intravascular device 102, pulling the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104.

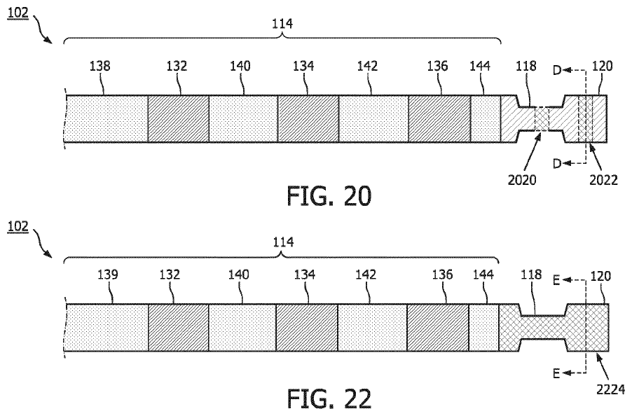

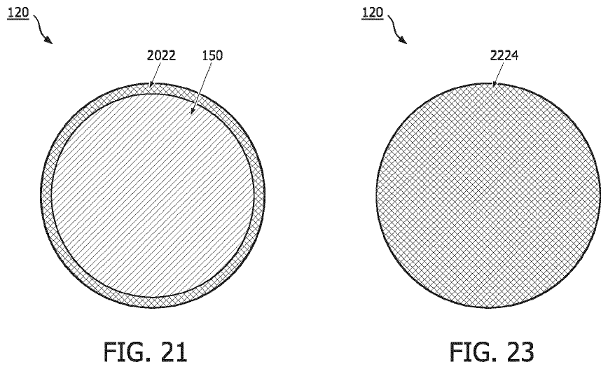

FIG. 20 is a diagrammatic cross-sectional view of the connection portion 114, the locking section 118, and the knob or retention section 120 of the flexible elongate member 106, according to aspects of the present disclosure. FIG. 21 illustrates a cross-sectional view of the knob or retention section 120 of the flexible elongate member 106 of FIG. 20, as seen along the lines of the section D-D taken therein, according to aspects of the present disclosure. FIGS. 20 and 21 include features similar to those described in FIGS. 10 and 11. In the embodiments of FIG. 20, the magnets 2020 and 2022 are formed in annular-ring shapes around a circumference of the metal core 150 of the locking section 118 and the knob or retention section 120, respectively. In the embodiments of FIG. 21, magnet 2022 is formed as an annular-ring shape around a circumference of the metal core 150 of the knob or retention section 120. In some embodiments, magnets 2022, when combined with magnet 2020 and when combined with magnets in the connector 104, described hereafter, pull the flexible elongate member 106 down into the recess 208 so flexible elongate member 106 is positioned inside recess 208 of the connector 140, locking section 118 is positioned aligned with slot 201, and conductive portions 132, 134 and, 136 are longitudinally aligned with the split, open-comb electrical contacts 932A, 932B, 934A, 934B, and 936 of connector 104. In some embodiments, magnets 2022, when combined with magnet 2020, are attracted to metal in connector 104, pulling the flexible elongate member 106 down into the recess 208 so flexible elongate member 106 is positioned inside recess 208 of the connector 140, locking section 118 is positioned aligned with slot 201, and conductive portions 132, 134 and, 136 are longitudinally aligned with the split, open-comb electrical contacts 932A, 932B, 934A, 934B, and 936 of connector 104.

FIG. 22 is a diagrammatic cross-sectional view of the connection portion 114, the locking section 118, and the knob or retention section 120 of the flexible elongate member 106, according to aspects of the present disclosure. FIG. 23 illustrates a cross-sectional view of the knob or retention section 120 of the flexible elongate member 106 of FIG. 22, as seen along the lines of the section E-E taken therein, according to aspects of the present disclosure. FIGS. 22 and 23 include features similar to those described in FIGS. 10 and 11. In the embodiments of FIG. 22, magnet 224 is formed to have the locking section 118 and the knob or retention section 120 and is disposed at the proximal portion 109 of the flexible elongate member 106. In the embodiments of FIG. 23, magnet 2224 is a solid magnet. In some embodiments, magnet 2224, when combined with magnets in the connector 104, described hereafter, pulls the flexible elongate member 106 down into the recess 208 so flexible elongate member 106 is positioned inside recess 208 of the connector 140, locking section 118 is positioned aligned with slot 201, and conductive portions 132, 134 and, 136 are longitudinally aligned with the split, open-comb electrical contacts 932A, 932B, 934A, 934B, and 936 of connector 104. In some embodiments, magnet 2224 is attracted to metal in connector 104, pulling the flexible elongate member 106 down into the recess 208 so flexible elongate member 106 is positioned inside recess 208 of the connector 140, locking section 118 is positioned aligned with slot 201, and conductive portions 132, 134 and, 136 are longitudinally aligned with the split, open-comb electrical contacts 932A, 932B, 934A, 934B, and 936 of connector 104.

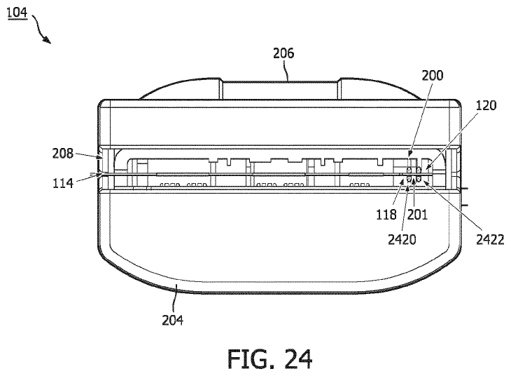

FIG. 24 is a diagrammatic top view of the connector 104 of the intravascular system 100, while the connector is in an open position, according to aspects of the present disclosure. FIG. 24 include features similar to those described in FIG. 14. In the embodiments of FIG. 24, one or more magnets may be positioned within connector 104 such that magnet 2420 is disposed below locking clip 200 and magnet 2422 is disposed proximate to locking clip 200. In some embodiments, magnets 2420 and 2422 have an opposite polarity to that of magnets 2020 and 2022 in the connection portion 114 of the flexible elongate member 106. Thus, in some embodiments, a magnetic attraction between magnets 2420 and 2422 and magnets 2020 and 2022 pulls the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, magnet 2422 has an opposite polarity to that of magnet 2224 in the connection portion 114 of the flexible elongate member 106. Thus, in some embodiments, a magnetic attraction between magnet 2422 and magnets 2224 pulls the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, magnets 2420 and/or 2422 are attracted to metal in intravascular device 102, pulling the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104.

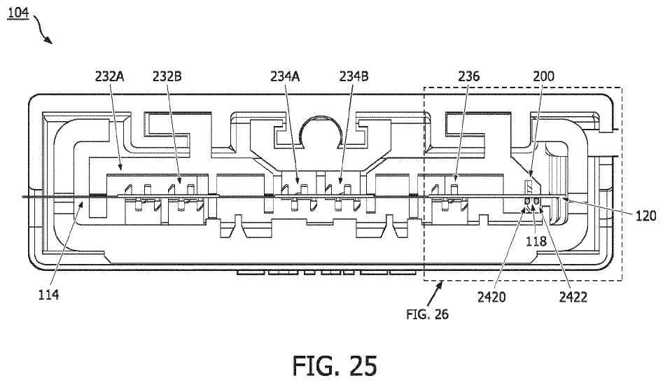

FIG. 25 is diagrammatic cross-sectional side view of connector 104, according to aspects of the present disclosure. FIG. 25 include features similar to those described in FIG. 15. In some embodiments, magnets 2420 and 2422 have an opposite polarity to that of magnets 2020 and 2022 in the connection portion 114 of the flexible elongate member 106. Thus, a magnetic attraction between magnets 2420 and 2422 and magnets 2020 and 2022 pulls the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, magnets 2420 and/or 2422 are attracted to metal in intravascular device 102, pulling the flexible elongate member 106 down into the recess 208 so flexible elongate member 106 is positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104.

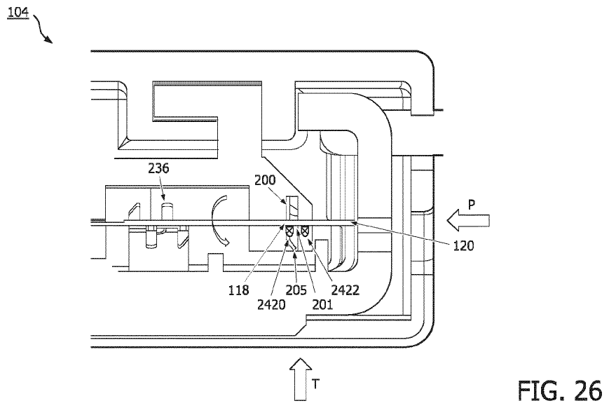

To further illustrate the details of locking clip 200, the proximal portion of connector 104 is enlarged and shown in FIG. 26, according to aspects of the present disclosure. In some embodiments, locking clip 200 includes a top portion 205 that tilts proximally at a tilt angle. The tilt angle is defined between the plane where the top portion 205 resides and the plane where the rest of locking clip 200 resides. In some embodiments, the tilt angle is between 10 and 90 degrees. Once locking section 118 is received in slit 201 of locking clip 200, the proximally tilting top portion 205 prevents locking section 118 from slipping upwards out of the slit 201. Advantageously, the engagement of locking section 118 and slit 201 of locking clip 200 ensure reliable electrical connection between conductive portions 132, 134, and 136 of connection portion 114 and split, open-comb electrical contacts 232A, 232B, 234A, 234B, and 236 in connector 104. Again, magnet 2420 is disposed below locking clip 200 and magnet 2422 is disposed proximate to locking clip 200 such that, in some embodiments, magnets 2020 and 2022 and/or magnet 2224 are attracted to magnet 2420 and/or magnet 2422, pulling flexible elongate member 106 down into the recess 208 and positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, metal in intravascular device 102 is attracted to magnet 2420 and/or magnet 2422, pulling flexible elongate member 106 down into the recess 208 and positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104.

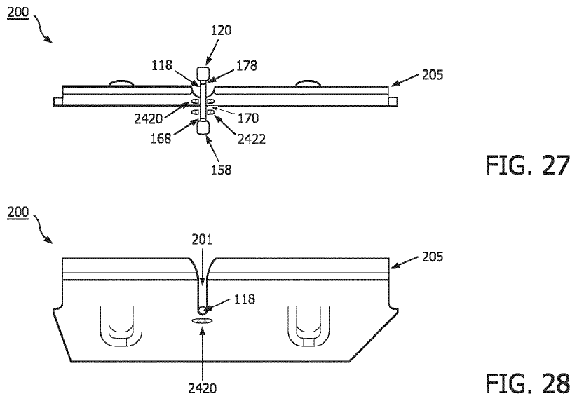

FIG. 27 is a diagrammatic top view of locking clip 200 from direction T shown in FIG. 26, according to aspects of the present disclosure. In some embodiments, locking section 118 includes distal subsection 168, middle subsection 170, and proximal subsection 178. When locking section 118 is received within slit 201 of locking clip 200, the movement of locking section 118 relative to locking clip 200 is limited to the length of the middle subsection 170. The proximally tilting top portion 205 may engage the proximal portion 178 and section 120 to prevent locking section 118 from slipping out of the slit 201. Again, magnet 2420 is disposed below locking clip 200 and magnet 2422 is disposed proximate to locking clip 200 such that, in some embodiments, magnets 2020 and 2022 and/or magnet 2224 are attracted to magnet 2420 and/or magnet 2422, pulling flexible elongate member 106 down into the recess 208 and positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiments, metal in intravascular device 102 is attracted to magnet 2420 and/or magnet 2422, pulling flexible elongate member 106 down into the recess 208 and positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104.

FIG. 28 is a diagrammatic proximal view of locking clip 200 from direction P shown in FIG. 26, according to aspects of the present disclosure. In some instances, locking section 118 is received within slit 201 of locking clip 200. The top portion 205 tilts proximally at the tilt angle. In some embodiments, slit 201 extends halfway through the height of locking clip 200. In some embodiment, magnet 2020 in the flexible elongate member 106 are attracted to magnet 2420 disposed below locking clip 200 so that flexible elongate member 106 is pulled down into the recess 208 and positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104. In some embodiment, metal in the intravascular device 102 is attracted to magnet 2420 disposed below locking clip 200 so that flexible elongate member 106 is pulled down into the recess 208 and positioned inside recess 208 of the connector 140, locking section 118 is positioned aligned with slot 201, and conductive portions 132, 134, and 136 are longitudinally aligned with the split, open-comb electrical contacts 932A, 932B, 934A, 934B, and 936 of connector 104.

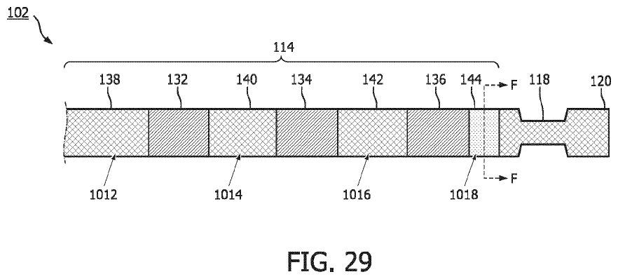

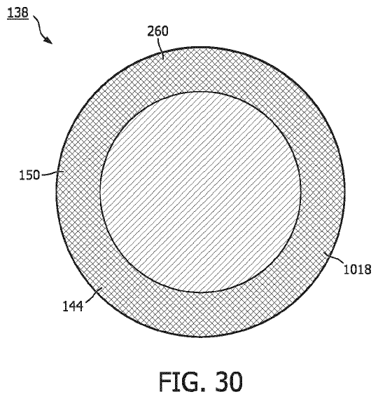

FIG. 29 is a diagrammatic cross-sectional view of the connection portion 114, the locking section 118, and the knob or retention section 120 of the flexible elongate member 106, according to aspects of the present disclosure. FIG. 29 can include aspects similar to those described with respect to FIGS. 10, 12A, 12B, 16, 20, and/or 22. FIG. 30 illustrates a cross-sectional view of the connection portion 114 of the flexible elongate member 106 of FIG. 29, as seen along the lines of the section F-F taken therein, according to aspects of the present disclosure. FIG. 30 can include aspects similar to those described with respect to FIGS. 11, 13, 17, 21, and/or 23. FIGS. 29 and 30 illustrate that the conductive portions 138, 140, 142, 144 and the magnets 1012, 1014, 1016, 1018 can be the same respective component. For example, the conductive band that is used to transmit electrical signals is a magnet. That is, the conductive portion 138 and the magnet 1012 are one same component, the conductive portion 140 and the magnet 1014 are one same component, the conductive portion 142 and the magnet 1016 are one same component, and/or the conductive portion 144 and the magnet 1018 are one same component. In some instances, as shown in FIG. 30, the one same component (the conductive portion 144 and the magnet 1018) completely surrounds and contacts the core wire 150. In some instances, a space is radially provided between the one same component (the conductive portion 144 and the magnet 1018) and the core wire 150.

Accordingly, it may be seen that the incorporating magnets at strategic locations in the connection portion of the flexible elongate member of the intravascular device and/or the connector pulls the flexible elongate member down into the recess of the connector and positions the flexible elongate member such that the locking section is aligned within the slot and the conductive portions are longitudinally aligned with the split, open-comb electrical contacts of the connector. This may tend to provide correct usage of the locking core feature within the connector and thus, reduce the risk of misconnection and damage to the proximal end of the flexible elongate member.

The logical operations making up the embodiments of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use electrical and electronic devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of aspects of the present disclosure. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary embodiments of aspects of the present disclosure defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

What is claimed is:

1. A system, comprising:
an intravascular guidewire comprising:
a flexible elongate member configured to be positioned within a blood vessel of a patient, wherein the flexible elongate member comprises a proximal portion and a distal portion;
a sensor disposed at the distal portion of the flexible elongate member, wherein the sensor is configured to obtain medical data related to the blood vessel while the flexible elongate member is positioned within the blood vessel;
a guidewire electrical contact disposed at the proximal portion of the flexible elongate member, wherein the guidewire electrical contact is in electrical communication with the sensor;
a connector configured to be removably coupled to the intravascular guidewire, wherein the connector comprises:
a slot configured to receive the proximal portion of the flexible elongate member; and
a connector electrical contact configured to contact the guidewire electrical contact to establish electrical communication with the sensor when the proximal portion of the flexible elongate member is received within the slot,
wherein at least one of the proximal portion of the guidewire or the connector comprises a magnet,
wherein the magnet is configured to facilitate at least one of:
positioning the proximal portion of the flexible elongate member within the slot; or
aligning the guidewire electrical contact and the connector electrical contact.

2. The system of claim 1,
wherein the connector comprises the magnet, and
wherein the magnet is disposed below the slot.

3. The system of claim 1,
wherein the connector comprises the magnet,
wherein the magnet is disposed proximate to the connector electrical contact.

4. The system of claim 3, wherein the magnet is aligned with the connector electrical contact.

5. The system of claim 3, wherein the magnet is offset from the connector electrical contact.

6. The system of claim 3,
wherein the connector comprises a plurality of connector electrical contacts,
wherein the magnet is disposed between the plurality of connector electrical contacts.

7. The system of claim 3,
wherein the connector comprises a plurality of magnets and a plurality of connector electrical contacts,
wherein the plurality of magnets is disposed proximate to the plurality of connector electrical contacts.

8. The system of claim 1,
wherein the proximal portion of the flexible elongate member comprises a first section with a first diameter and a second section with a second diameter less than the first diameter,
wherein the connector comprises:
the magnet; and
a locking feature configured to engage the second diameter of the second section,
wherein the magnet is disposed proximate to locking feature.

9. The system of claim 8, wherein the magnet is disposed proximal of the locking feature.

10. The system of claim 1,
wherein the connector comprises the magnet,
wherein the proximal portion of the flexible elongate member comprises a further magnet,
wherein the magnet and further magnet are arranged such that opposite polarities of the magnet and further magnet attract one another.

11. The system of claim 10, wherein the further magnet is disposed proximate to the guidewire electrical contact.

12. The system of claim 11, wherein the further magnet is aligned with the guidewire electrical contact.

13. The system of claim 11, wherein the further magnet is offset from the guidewire electrical contact.

14. The system of claim 10,
wherein the proximal portion of the flexible elongate member terminates at a proximal end,
wherein the further magnet is proximate to the proximal end.

15. A system, comprising:
an intravascular guidewire comprising:
a flexible elongate member configured to be positioned within a blood vessel of a patient, wherein the flexible elongate member comprises a proximal portion and a distal portion;
at least one of a pressure sensor or a flow sensor disposed at the distal portion of the flexible elongate member and configured to obtain at least one of pressure data or flow data related to the blood vessel while the flexible elongate member is positioned within the blood vessel;
a guidewire electrical contact disposed at the proximal portion of the flexible elongate member, wherein the guidewire electrical contact is in electrical communication with at least one of the pressure sensor or the flow sensor;
a connector configured to be removably coupled to the intravascular guidewire, wherein the connector comprises:
a slot configured to receive the proximal portion of the flexible elongate member; and
a connector electrical contact configured to contact the guidewire electrical contact to establish electrical communication with at least one of the pressure sensor or the flow sensor when the proximal portion of the flexible elongate member is received within the slot,
wherein at least one of:
the proximal portion of the guidewire comprises a magnet proximate to the guidewire electrical contact; or
the connector comprises a further magnet proximate to the connector electrical contact,
wherein at least one of the magnet or the further magnet is configured to facilitate aligning the guidewire electrical contact and the connector electrical contact.

16. A system, comprising:
an intravascular guidewire comprising:
a flexible elongate member configured to be positioned within a blood vessel of a patient, wherein the flexible elongate member comprises a proximal portion and a distal portion, wherein the proximal portion comprises a first section with a first diameter and a second section with a second diameter less than the first diameter;
at least one of a pressure sensor or a flow sensor disposed at the distal portion of the flexible elongate member and configured to obtain at least one of pressure data or flow data related to the blood vessel while the flexible elongate member is positioned within the blood vessel;
a guidewire electrical contact disposed at the proximal portion of the flexible elongate member, wherein the guidewire electrical contact is in electrical communication with at least one of the pressure sensor or the flow sensor;
a connector configured to be removably coupled to the intravascular guidewire, wherein the connector comprises:
a slot configured to receive the proximal portion of the flexible elongate member;
a connector electrical contact configured to contact the guidewire electrical contact to establish electrical communication with at least one of the pressure sensor or the flow sensor when the proximal portion of the flexible elongate member is received within the slot; and
a locking feature configured to engage the second diameter of the second section, and
wherein at least one of:
the proximal portion of the guidewire comprises a magnet; or
the connector comprises a further magnet proximate to the locking feature,
wherein at least one of the magnet or the further magnet is configured to facilitate positioning the proximal portion of the flexible elongate member within the slot.
